(19) United States
(12) Patent Application Publication
(10) Pub. No.: US 2025/0281716 A1
(43) Pub. Date: Sep. 11, 2025

(54) Isolation Therapy Device for Sensory Reduction and Neurological Applications

(71) Applicant: OASIS SYSTEMS, Pittsburgh, PA (US)

(72) Inventor: Fiona Katharine McCarthy, Pittsburgh, PA (US)

(73) Assignee: OASIS SYSTEMS, Pittsburgh, PA (US)

(21) Appl. No.: 19/092,696

(22) Filed: Mar. 27, 2025

(51) Int. Cl. A61M 21/00

(52) U.S. Cl. CPC A61M 21/0094; A61M 2021/0016; A61M 2021/0022; A61M 2021/0027; A61M 2021/0044; A61M 2021/0066; A61M 2230/10

(57) ABSTRACT

An isolation therapy device worn on the head for reducing sensory input and delivering therapeutic treatment for neurological and psychological conditions, including anxiety, PTSD, autism, cognitive decline, and concussion. The device comprises a multi-layer construction including: an innermost comfort layer, an intermediate adjustable compression or suspension layer, and an outer fabric shell layer. The device further includes optional modular systems for sensory reduction and therapeutic enhancement, including modules for visual and auditory isolation, aromatherapy, heating, cooling, vibration, and EEG monitoring. Modular components may be removable, adjustable, and customizable. The device optionally integrates smart sensors for biometric monitoring and real-time feedback. A digital application may be used in conjunction with the device to enable control, tracking, and personalization of therapy parameters. The modular nature allows for individualized treatment protocols and adaptability across a range of conditions.

### SUMMARY OF THE INVENTION

The invention provides an isolation therapy device worn on the head, designed to reduce sensory stimuli, provide a soft sensation through compression, and deliver therapeutic features via modular systems. The device may comprise the following modular systems:
An adjustable soft compression system 100, similar to a hard hat suspension system but made of lightweight and/or soft materials, adjustable compression via an adjustment knob 106, a ratchet system 107 to provide compression, or adjustable compression via an electronic smart component to shorten and lengthen the suspension straps 105.
A sound reduction system 400 that covers the ears, reduces sound, and optionally integrates speakers, and/or active or passive noise cancelation.
A sight reduction system 700 that provides darkness, with optional integrated heating, cooling, massage, and/or vibration, and the ability to easily attach and detach externally via a clasp 704.
A smell system 900 that delivers scent using swappable, scent-infused inserts 901, and/or sticks.
A heating system 301 and/or cooling system 301 and may use reusable cooling pads and/or reusable heating pads 301 and may be integrated into the innermost layer 300.
Sensor arrays may be for adjusting tension of the suspension straps 401, may be for generating a vibration via a vibration coin 1206, may measure brainwaves, and/or biometrics, and/or EEG via a brainwave pad 1301. Optionally the sensors may be linked to an app and/or AI computer to generate therapeutic modes for the user like (e.g., “Concussion Mode”, “Anxiety Mode”, “Sensory Mode”).

The isolation therapy device 50 may have a three-layer construction:
1. An innermost layer 300: the innermost layer may be comprised of a cloth hat like layer, may be comprised of a layer that has integrated heating and or cooling pads 301, may be comprised of a gel like layer, may be comprised of removable, and/or washable integrated ear pads 302 that may act as the sound reduction system 400. May also be soft and cushioning so that a person can lay their head down and feel comfortable.
innermost layer may be comprised of a chin strap 303 or may be chin strap free 651.
2. An adjustable soft compression layer 100: provides a soft and comfortable feel, may be comprised of soft to the touch fabrics, light, soft, and/or flexible plastic parts, adjustable head tension, and/or an adjustment knob 106.
3. An outermost layer 200: may be comprised of a breathable, stretchable, elastic-like, and/or flexible fabric, that my be aesthetically customizable. May also be soft and cushioning so that a person can lay their head down and feel comfortable.

The isolation therapy device 50 may have a two-layer construction:
1. A first layer that is an innermost layer 300: the innermost layer may be comprised of a cloth hat like layer, may be comprised of a layer that has integrated heating and or cooling pads 301, may be comprised of a gel like layer, may be comprised of removable, and/or washable integrated ear pads 302 that may act as the sound reduction system 400. The innermost layer may be comprised of a chin strap 303 or may be chin strap free 651.
2. A second layer that combines the adjustable soft compression layer 100 and the outermost layer 200 into a single second layer.

The isolation therapy device 50 may have a fully integrated construction where the innermost layer 300, the adjustable soft compression layer 100, and the outermost layer 200 are integrated into a single layer.

### BRIEF DESCRIPTION OF THE DRAWINGS

FIG. 1A: Depicts an isolation therapy device 50

FIG. 1B: Depicts an adjustable soft compression layer 100

FIG. 1C: Depicts an outermost layer 200

FIG. 1D: Depicts an innermost layer 300

FIG. 2A: Depicts a sound reduction system 400

FIG. 2B: Depicts a removable ear pad 500

FIG. 3A: Depicts an isolation therapy device with a sound reduction system 600

FIG. 3B: Depicts a chin strap free isolation therapy device with a sound reduction system 650

FIG. 4A: Depicts a sight reduction system 700

FIG. 5A: Depicts an isolation therapy device with a sight reduction system 800

FIG. 5B: Depicts an isolation therapy device with a removable sight reduction system 850

FIG. 6A: Depicts a smell system 900, 1000

FIG. 7A: Depicts a sensor and/or vibration system 1200

FIG. 7B: Depicts a sensor and/or vibration coin assemblies 1300, 1400, 1500, 1600

### DETAILED DESCRIPTION OF THE INVENTION

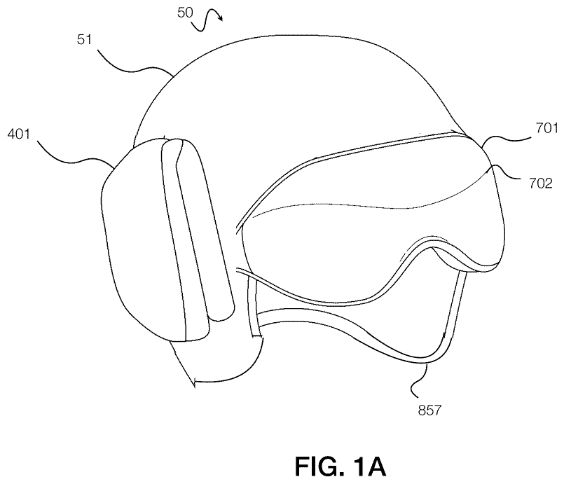

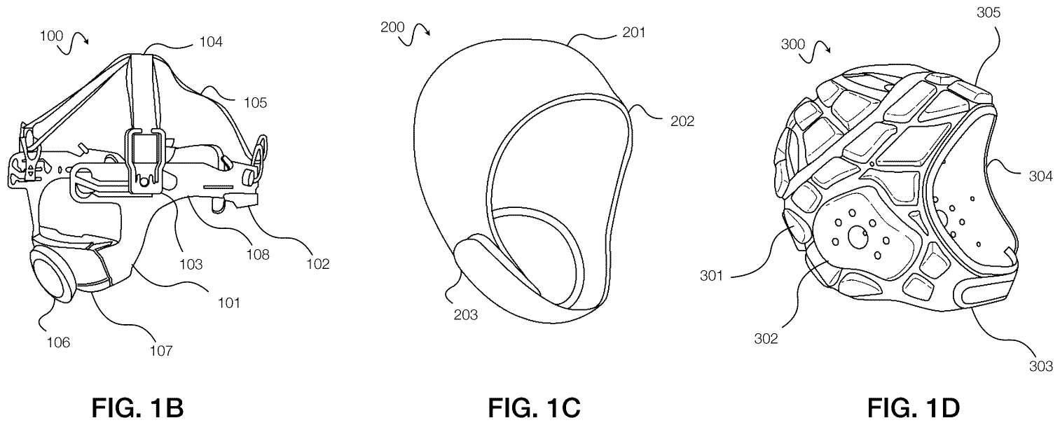

FIG. 1A depicts an isolation therapy device 50. An isolation therapy device 50 is worn on the head and shaped to accommodate the contours of the human skull. The construction of the device may be a three layer construction. In the three layer construction, isolation therapy device 50 comprises an outermost layer 200, an adjustable soft compression layer 100, and an innermost layer 300. The construction of the device may be a two layer construction. In the two layer construction, isolation therapy device 50 comprises a first layer that is comprised of an outermost layer 200, an second layer that is comprised of both the adjustable soft compression layer 100, and an innermost layer 300.

FIG. 1B depicts an adjustable soft compression layer 100. The adjustable soft compression layer 100, is inspired by hard-hat suspension systems but made from lightweight, soft materials. The adjustable soft compression layer 100 can provide compression independently of any other layer in the isolation therapy device 50. The adjustable soft compression layer 100 may be comprised of soft plastic, flexible plastic, cloth, fabric, lightweight plastic and/or padding. The adjustable soft compression layer 100 may be comprised of an upward extension part 101 that provides that system improved compression performance, a forehead headband 102 that provides comfortable compression around the head, a suspension strap-to-headband clasp 103 that connects the suspension strap 105 to the headband 108, a suspension center 104 that provides stability for each suspension strap 105 at the crown of the head and provides compression to the crown then the adjustable soft compression layer 100 is tightened, an adjustable knob 106 that works to tighten and loosen the soft compression layer 100 through a ratchet system 107. The adjustable knob 106 may be a knob shape, a disc shape, a lever, and slider, and button, and/or may be made to have a compression adjustment that is fully automated via an external source like an app on a mobile phone. The adjustable soft compression layer 100 may be comprised of suspension straps 105 adjustable straps that converge at the crown of the head at a suspension center 104. The adjustable soft compression layer 100 may be comprised of a backhead band 1203 that may provide additional compression and additional support for the ratchet system 107. The headband 108 may wrap around the circumference of the head. The headband 108 may be comprised of several parts in order to make it physically connected around the circumference of the head. The ratchet system may be constructed as a ratchet system 107, that has an adjustable knob 106 that can be turned clockwise or counter clockwise to tighten and loosen the level of compression. The adjustable soft compression layer 100 may have a number Nc of suspension straps 105 where Nc may equal 4, 6, 8, 10, 12, 14, 18, 20 or may be a number between 1 and 20, 20 and 40, 40 and 100, and 100 and 200.

The adjustable soft compression layer 100 may be engineered so that the isolation therapy device delivers specific distributed pressure to each of many preset contact points. The contact points may be set as an array of contact points where the adjustable soft compression layer 100 touches the head. Compression at each contact point may be optimized to deliver light pressure, moderate pressure, and/or high pressure on the head and/or at different points on the head. Very light compression and/or pressure may be an average force N on the head of 0 N to 20 N or an average pressure P on the head between 0 kPa and 1 kPa. Light compression and/or pressure may be an average force N on the head of 20 N to 50 N or an average pressure P on the head between 1 kPa and 3 kPa. Moderate compression and/or pressure may be an average force N on the head of 50 N to 100 N or an average pressure P on the head between 3 kPa and 7 kPa. High compression and or pressure may be an average force N on the head of 100 N to 200 N or an average pressure P on the head between 7 kPa and 15 kPa.

Pressure on the head P may be measured by standard methods and may have a kilopascal value between 0.5 kPa and 1 kPa, 1 kPa and 2 kPa, 2 kPa and 5 kPa, 5 kPa and 10 kPa, 10 kPa and 20 kPa, and/or greater than 20 kPa at any given measurement point. The force on the head at any single contact point may be measured by standard methods and have a Newton N value between 0 N and 1 N, 1 N and 3 N, 3 N and 5 N, 5 N and 10 N, 20 N and 50 N, 50 N and 100 N, 100 N and 200 N and/or greater than 200 N at any given measurement point.

The soft compression layer 100 may be optimized to deliver adjustable pressure P and/or force N to different locations of the head. The soft compression layer 100 may be comprised of tension control on part and/or straps to allow the user and/or software the ability to deliver specific pressure P and/or force N to specific locations on the head during use.

FIG. 1C depicts an outermost layer 200. The outermost layer 200 comprises a soft, breathable cover and may be comprised of fabric, elastic, stretchable fabric, printable fabric, colorful fabric, fabric with a logo and or symbol, washable fabric, exchangeable fabric, and/or customizable fabric.

FIG. 1D depicts an innermost layer 300. The innermost layer 305 serves as the comfort interface between the head and the compression system and may be comprised of memory foam, gel pads, or other padded materials. The innermost layer may be removable via velcro, snaps, and/or magnetic attachments. The innermost layers may also feature heating and/or cooling pads 301. The innermost layer 300, and/or heating pads, and/or cooling pads 301, may be comprised of sewn down blanket-type fabric, feathers, down feathers, pillow type material, cushion, fabric, padded fabric, pads, gel pads, fabric covered gel, fabric covered memory foam, memory foam, fabric covered cooling gel, cooling gel, fabric covered warming gel, and/or fabric-based pads, optionally microwaveable and/or refrigerable, and may be machine washable. The innermost layer 305 may be comprised of an ear pad 302 to reduce sound and improve comfort. The innermost layer may be comprised of a chin strap 303 or may be chin strap free. The chin strap 303 may connect through magnets, a clasp, a buckle, snaps, and/or velcro.

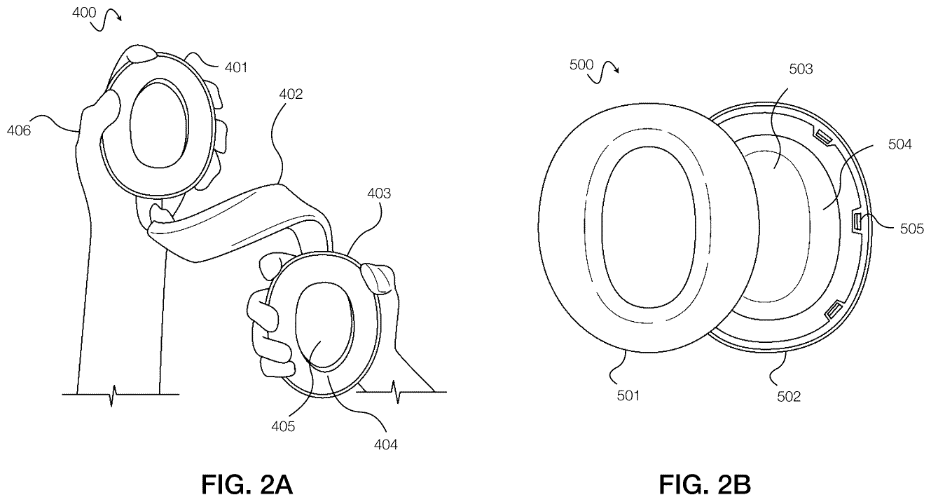

FIG. 2A depicts a sound reduction system 400. The sound reduction system 400 may be comprised of a left ear pad 401 and a right ear pad 403. The ear pad 401,403 may be comprised of a soft cushion 404 and an ear cavity 405 to provide sound reduction and comfort. The sound reduction system 400 may be comprised of a sound reduction system headband 402 that makes the sound reduction system 400 easily removable. The sound reduction system 400 may be integrated into the innermost layer 305 or the outermost layer 200 in a three layer construction. The sound reduction system 400 may be integrated into the second layer in a two layer construction. The sound reduction system 400 may be comprised of passive ear-muff style pads 401,402 and/or speakers, and/or active noise-canceling speakers. Optionally, a sound reduction system 400 can detect ambient noise levels and feed data to a mobile application. The sound reduction system 400 may reduce auditory stimuli.

FIG. 2B depicts a removable ear pad 500. Removable ear pads 500 may be comprised of removable ear pad cushions 501, a removable ear pad scaffold 502, an ear pad cavity 503, an ear pad cushion connector 504, and/or clips 505 for connecting the ear pad cushion 501 to the ear pad scaffold 502, and/or clips 505 to connect the removable ear pad 500 to the isolation therapy device 50.

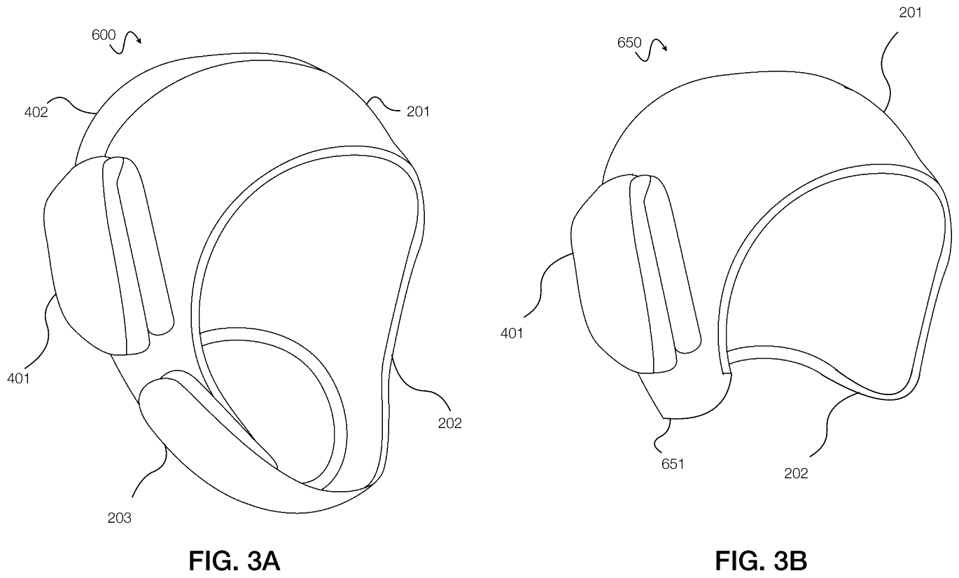

FIG. 3A depicts an isolation therapy device 50 with a sound reduction system 600 and FIG. 3B depicts a chin strap free isolation therapy device with a sound reduction system 650 where the figure shows the absence of a chin strap 651.

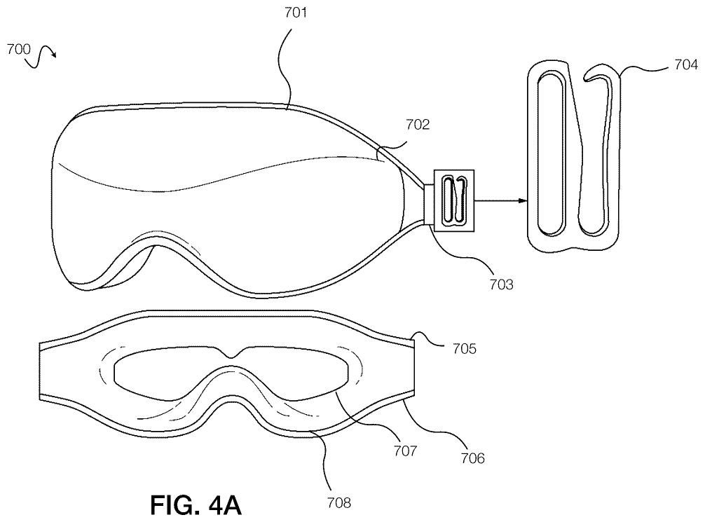

FIG. 4A depicts a sight reduction system 700. A sight reduction system may be comprised of a sight reduction system band 703 that may connect the eye mask 701 to the clasp 704 or may extend as a band to allow the eye mask 701 to be placed on the head. The outside of the eye mask 701 may be comprised of a pouch 702 for the smell system. The inside of the eye mask 701 may be comprised of a light seal 706, a cavity for the eyes 707, and/or a cushion 708. The sight reduction system 700 may be made easily removable by incorporating the eye mask 701 into a removable headband. The sight reduction system 700 may be removably attachable to the device and configured to block light.

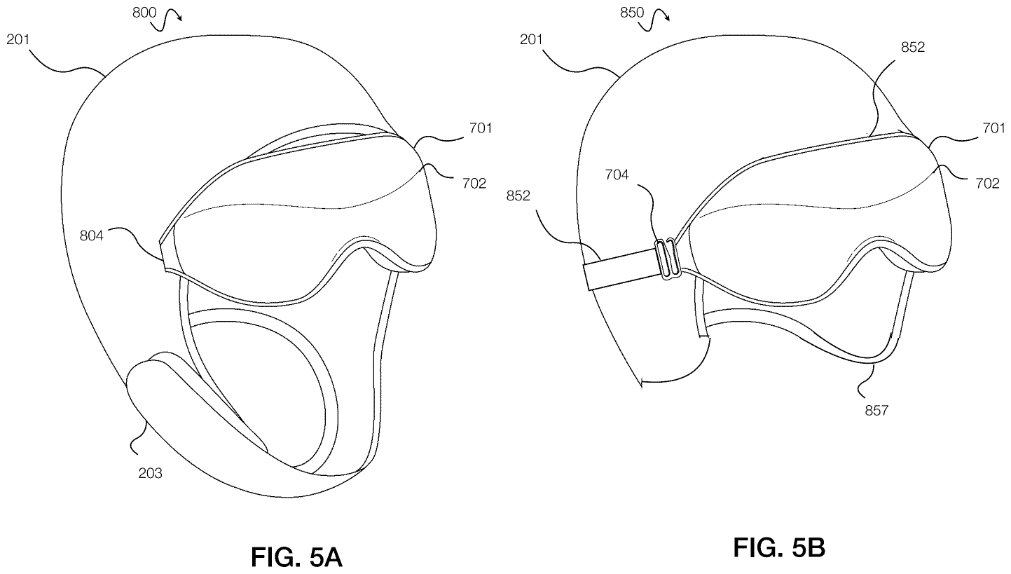

FIG. 5A depicts an isolation therapy device 50 with a sight reduction system 800. The sight reduction system 800 may be integrated into the outermost layer 201 or the sight reduction system 800 may be integrated into the innermost layer 305.

FIG. 5B: Depicts an isolation therapy device 50 with a removable sight reduction system 850. The sight reduction system 800 may be attached to the outermost layer 201 via a clasp 704 making it removable. The clasp 704 may be a clasp, a clip, a magnetic connector, a velcro connector and/or a button which makes it easy and convenient to remove the eye mask 701. The sight reduction system 800 may be attached to the innermost layer 201 or the second layer via a clasp 704 making it removable. The clasp 704 may be a clasp, a clip, a magnetic connector, a velcro connector and/or a button which makes it easy and convenient to remove the eye mask 701. The isolation therapy device 50 with a removable sight reduction system 850 may have a chin strap 203 or may be chin strap free 857. The sight reduction system 800, 850 may be comprise of a flexible mask designed to block light. It may be comprised of a massage/vibration features and/or thermal pads.

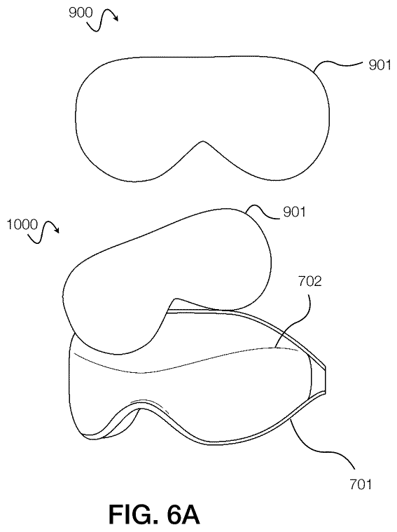

FIG. 6A depicts a smell system 900, 1000. A smell system may be comprised of a smell system insert 901. The smell system insert 901 may be an insert that contains a scent. The scent may be from perfume, essential oil, fragrance, therapeutic fragrance, essence, and/or oil. The smell system insert 901 may be reusable and/or refreshed after use. The smell system insert 901 may also be disposable. The smell system insert may be inserted into a pouch 702 in the eye mask 701. The pouch 702 and insert 901 may be in the form depicted in FIG. 6A. The pouch and the insert may be the form of a stick and stick shaped pouch not shown.

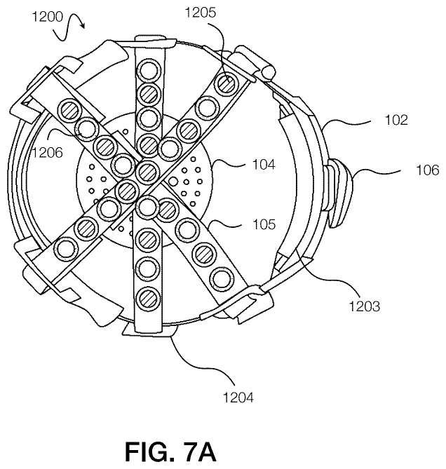

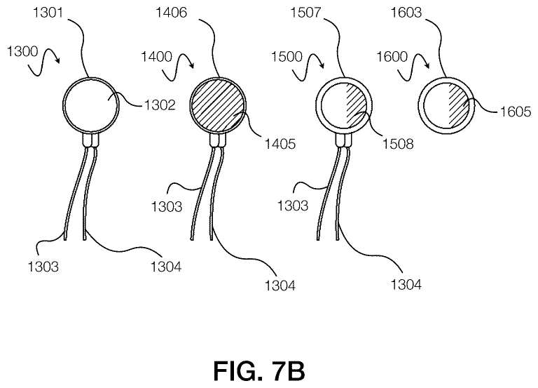

FIG. 7A depicts a sensor and/or vibration system 1200. The sensor and/or vibration system 1200 may be comprised of a suspension strap connector 1204, brainwave sensors 1205, and/or vibration coins 1206. The suspension strap connector 1204 may connect the suspension strap 105 to the headband and may provide structural support to improve compression. Brainwave sensors 1205 may be adhered to the suspension straps 105 in an array that allows the brainwave sensors 1205 to sense signals from the brain at different locations on the head. Alternatively, brainwave sensors 1205 may be adhered to the suspension straps 105 and protrude through the innermost layer such that they can be in direct contact with a variety of locations on the head, in an array that allows the brainwave sensors 1205 to sense signals from the brain at different locations on the head. Alternatively, brainwave sensors 1205 may be adhered to the innermost layer 105 in an array that allows the brainwave sensors 1205 to sense signals from the brain at different locations on the head. Vibration coins 1206 may be adhered to the suspension straps 105 in an array that allows vibration signals to be sent to different parts of the head. These vibration signals may be random, or may be preprogramed. The signal preprogram may be controlled by an application in an external device and may be an app in a mobile device. The levels of vibration may be modulated to achieve an improved user experience and/or a target brainwave response.

FIG. 7B: Depicts a sensor and/or vibration coin assemblies 1300, 1400, 1500, 1600. The vibration coin assembly 1300 may be comprised of a vibration pad 1301, a frame 1302, and data and/or electron wires 1303, 1304. The brainwave sensor assembly 1400 may be comprised of a brainwave pad 1405, a frame 1406, and data and/or electron wires 1303, 1304. Dual vibration/brainwave sensor assembly 1500 may be comprised of a dual pad 1508, a frame 1603, and data and/or electron wires 1303, 1304.

Either the vibration assembly, the brainwave sensor assembly, or the dual assembly may be wireless 1600 and may have a wireless pad 1605 and/or a wireless frame 1603 where power is in a rechargeable battery within the assembly and data is transported wirelessly to and from the wireless assembly 1600. Each brainwave sensor 1400 may measure any one of the following; brainwaves, EEG, electroencephalography, delta waves, theta waves, alpha waves, beta waves, gamma waves, Mu waves, SMR, sensorimotor rhythm, REM, rapid eye movements, non-REM, combinations of brain waves, neurofeedback, biofeedback, brain activity, qEEG, quantitative electroencephalography, EOG, electrooculography, EMG, electromyography, hdEEG, high density electroencephalogram, MEG, magnetoencephalography, OPM, optically-pumped magnetometers, fNIRS, functional near-infrared spectroscopy, fUS, functional ultrasound brain imaging, skin temperature, heart rate, cardiac data, ECG, EKG, electrocardiogramathing rate, respiratory rate, breath depth, blood oxygen levels, blood pressure, sweat composition, hydration level, skin impedance, skin resistance, and/or glucose levels. This data can be integrated into a device and/or computer for use to improve consumer experience, monitor consumer experience, improve the device, and/or improve user outcomes. Vibration coins 1300, 1500 may be integrated into the soft compression layer 100 such that they are positioned in specific locations on the head and may be activated in patterns based on user preference or therapeutic mode. The brainwave sensor 1400 array may be designed to position the appropriate sensor at the appropriate location on the head. This brainwave sensor 1400 array design approach may allow the user to get more diverse data from a variety of different types of sensors at a variety of different locations on the head. Such arrays may result in improved biometric data collection. A sensor array may consist of a number of sensors Ns. Where Ns may be equal to any number between 1 and 10,000 and may be equal to a number between 5 and 10, 10 and 25, 25 and 50, 50 and 100, 100 and 500, 500 and 1,000, 1,000 and 5,000, and 5,000 and 10,000. Brainwave sensors 1400 may be integrated into the sight reduction system 700 to expand the coverage of sensors to additional locations on the head. Brainwave sensors 1400 may be integrated into the sound reduction system 700 to expand the coverage of sensors to additional locations on the head. The vibration coin assemblies 1300 may be individually addressable to provide programed vibration sensations and/or vibration sensations in coordination with brainwave sensor feedback.

A “lightweight isolation therapy device” means an isolation therapy device 50 that has a weight less than 20 ounces. A “very lightweight isolation therapy device” means an isolation therapy device 50 that has a weight less than 15 ounces. An “ultra lightweight isolation therapy device” means an isolation therapy device 50 that has a weight less than 10 ounces. An “extremely lightweight isolation therapy device” means an isolation therapy device 50 that has a weight less than 7 ounces. An “amazingly lightweight isolation therapy device” means an isolation therapy device 50 that has a weight less than 4 ounces.

To describe location on the head with clarity; left and right refer to the person's own sides (not viewer-facing), Anterior=Front, Posterior=Back, Superior=Top, Inferior=Bottom or below, Medial=Toward the midline and Lateral=Toward the side. The head can be described as to have: a Frontal Region (Forehead) with a Left Frontal Eminence: Upper left forehead, a Right Frontal Eminence: Upper right forehead, a Glabella: Center point between eyebrows, a Left Supraorbital Area: Above the left eyebrow, a Right Supraorbital Area: Above the right eyebrow, a Left Medial Forehead: Between glabella and left eminence, a Right Medial Forehead: Between glabella and right eminence; Temporal Regions with a Left Temple (Left Temporal Fossa): Above and slightly in front of the left ear, a Right Temple (Right Temporal Fossa): Above and slightly in front of the right ear, a Left Anterior Temporal: Toward the front edge of the left temple, a Right Posterior Temporal: Toward the back edge of the right temple; Parietal Region (Top/Sides of Head) with a Left Parietal: Upper left side of the skull, a Right Parietal: Upper right side of the skull, a Mid-Parietal Line: Runs along the sagittal suture (midline), a Left Superior Parietal: Higher part of the left side, a Left Inferior Parietal: Lower part of the left side (near ear level); Vertex (Crown of Head) with a Midline Vertex: Exact top center of the head (highest point), a Left Crown Region: Slightly to the left of vertex, a Right Crown Region: Slightly to the right of vertex, a Anterior Vertex: Toward forehead side, Posterior Vertex: Toward the back of the head; Occipital Region (Back of Head) with a Left Occipital: Back left side of the head, a Right Occipital: Back right side of the head, a Mid-Occipital Ridge: Center back ridge (external occipital protuberance), a Superior Occipital: Upper back of the skull, a Inferior Occipital: Near base of the skull/top of neck, a Left Posterolateral Occipital: Diagonally left, lower back; Nape/Suboccipital Region with a Left Nape: Left base of the head above neck, a Right Nape: Right base of the head above neck, a Central Nape (Midline Suboccipital): Directly at midline below occiput, an Upper Nape: Closer to the skull a Lower Nape: Transition area to upper neck; Auricular Region (Ears & Surroundings), a Left Auricular: Left ear and area just around it, a Right Auricular: Right ear and surrounding skin, a Preauricular: In front of ear, a Postauricular: Behind ear, a Superior Auricular: Upper edge of ear a Inferior Auricular: Earlobe and below; Facial-Adjacent Regions (relevant to headgear interfaces), a Zygomatic Arch (Left/Right): Cheekbone area, just in front of ear, a Mandibular Angle (Left/Right): Angle where jaw curves upward, a Temporal Line: Arching ridge where temporalis muscle connects (curves over parietal bone).

Alternately, to describe locations on the head with clarity, the Electrode Placement System-The International 10-20 System, which is a standardized system and familiar to one skilled in the art, may be used to describe locations on the head for placement of vibration coins, sensors, and/or for areas to apply pressure. Locations are determined based on the distances between specific anatomical landmarks of the skull, including the nasion (the indentation between the forehead and nose), the inion (the bony bump at the back of the skull), and the left and right preauricular points (just above the ears). Locations are described as intervals of either 10% or 20% of the total distance between these landmarks, forming a grid-like structure over the scalp. Each location site is labeled using a combination of a letter and a number: The letter refers to the underlying brain region: Fp=Frontal pole (forehead region), F=Frontal lobe, C=Central region (corresponding roughly to the motor cortex), P=Parietal lobe, T=Temporal lobe, O=Occipital lobe. The number indicates the hemisphere: Odd numbers (1, 3, 5, 7) are on the left hemisphere, Even numbers (2, 4, 6, 8) are on the right hemisphere, “Z” (zero) designates a position along the midline of the scalp. For example: Fp1 is located over the left frontal pole, just above the left eye. F3 is over the left frontal lobe. Cz is centered on the top of the head, along the midline. O1 is over the left occipital lobe, at the back of the head.

Correlation between brainwave measurements and symptoms from disorders are well known. The isolation therapy device 50 may be used to measure, monitor, and/or treat a wide variety of symptoms and/or disorders including but not limited to; emotional dysregulation, affective disorders, affective symptoms, mood disorders, mood symptoms, psychosocial symptoms, psychosocial disorders, anxiety, neurological disorder, neurodegenerative disorder, cognitive disorder, autism, autism spectrum disorder, dementia, Alzheimer's disease, Parkinson's disease, Huntington's disease, frontotemporal dementia, ALS, Lewy body dementia, multiple system atrophy, mild cognitive impairment, delirium, amnestic disorder, vascular dementia, mixed dementia, Parkinson's disease dementia, frontal lobe cognitive disorder, cognitive impairment due to brain injury, cognitive impairment due to stroke and/or concussion. The brainwave sensor 1400 array may be configured generally, and/or specifically, for example specific brainwave sensors 1400 and or vibration coins 1206 in specific places in the array so that may be in close proximity to a specific part of the head and may collect data, deliver sounds, and/or deliver vibrations in a manner that the isolation therapy device 50 may be used to detect, monitor and/or treat symptoms and/or disorders. Furthermore, data from the brainwave sensors 1400 may be used to identify new correlations between brainwave sensor 50 outputs and and symptoms, may be used to better measure baseline brainwave sensor 1400 activity, may result in routine use of the isolation therapy device 50, may result in new treatments, and/or may be used to monitor brainwave sensor 1400 data over time.

In a first example, the isolation therapy device 50 may be used to detect, monitor, and/or treat symptoms associated with a concussion. Brainwave sensors 1400 may be configured to measure EEG (electroencephalography) and may offer a way to monitor how one's brain is recovering from a concussion by measuring the brain's electrical activity across different frequency bands and in response to specific stimuli. The specific stimuli may be stimuli from the vibration coins 1206. Vibration coins 1206 may be placed in specific parts to the sensor array in order to produce a stimuli in specific parts of the head. Brainwave sensors 1400 may be placed in specific parts of the sensor array in order to measure signals from specific parts of the head. Two of the most useful EEG-based markers for concussion are P300 evoked potentials and the power levels in frequency bands like alpha and theta. The P300 waveform appears around 300 milliseconds (ms) after a person detects an important stimulus, like feeling a vibration or a sequence of vibrations and/or hearing a rare tone and/or a rare tone in a sequence of beeps. After a concussion, the P300 response is often delayed and weaker. For instance if a healthy brain shows a P300 latency of around 300 milliseconds and an amplitude (signal strength) between 10 microvolts and 20 microvolts. After a concussion, latency may increase, for example, to between 350 milliseconds and 450 milliseconds, and amplitude may drop, for example, to between 5 microvolts and 10 microvolts indicating slower and less coordinated processing. As recovery progresses, the latency should decrease toward normal levels, for example, toward 300 milliseconds and amplitude should rise again toward normal levels, for example toward between 10 microvolts and 20 microvolts. Additionally, a general resting-state EEG pattern may detect abnormal activity in the theta and alpha bands after a concussion. Theta waves (between 4 hertz and 8 hertz), are normally active during drowsiness or early learning, and tend to be abnormally elevated post-concussion. For example, theta power may start high, around 40 microvolts squared, and should gradually drop back to 25 microvolts squared or less as the brain heals. Alpha waves (between 8 hertz and 13 hertz), associated with calm wakefulness and mental readiness, are usually reduced after a concussion. Alpha power might begin as low as 15 microvolts squared, then increase steadily toward 30 microvolts squared during recovery. A common indicator of concussion is the theta-to-alpha power ratio: a high ratio, for example 2.67, signals dysfunction, and as you recover, that ratio should drop below 1.0. The isolation therapy device may track recovery, by measuring EEG periodically, for instance, periodically, daily, every other day, weekly, monthly, periodically through the week, periodically through the month, periodically through the year, and/or periodically over a period of years depending on the symptom and/or brainwave that is being monitored. Early on, you may receive data showing, for example: Alpha power starting at 15 microvolts squared and increasing each week (to 20 microvolts squared, 25 microvolts squared, and finally 30 microvolts squared). Theta power dropping from 40 microvolts² down to 25 microvolts squared. The theta/alpha ratio dropping from 2.67 (week 1) to 0.83 (week 4). P300 latency decreasing from 410 milliseconds to 300 milliseconds. P300 amplitude increasing from 6 microvolts to 12 microvolts. These trends indicate a return to normal electrical patterns and cognitive processing speed.

In a second example, the isolation therapy device 50 may be used to detect, monitor, and/or treat symptoms associated with cognitive decline, for example from Alzheimer's disease. Electroencephalography (EEG) data from brainwave sensors 1400 may be used to detect, monitor and/or treat neural dysfunction and cognitive decline in individuals with Alzheimer's disease by measuring the brain's electrical activity. As Alzheimer's progresses, changes in both spontaneous EEG rhythms and event-related potentials (ERPs) can reveal underlying impairments in cognitive processing, attention, and memory. One of the most consistent findings in Alzheimer's is a shift toward slower EEG activity. This is reflected in an increase in delta and theta waves and a decrease in alpha and beta waves, a phenomenon often referred to as EEG slowing. In early stages of Alzheimer's, theta power, which is between 4 hertz and 8 hertz, tends to increase significantly, often rising above 30 microvolts squared, indicating disrupted cortical activity and impaired attention. At the same time, alpha power, which is between 8 hertz and 13 hertz, which is normally dominant during resting wakefulness, often drops below 20 microvolts squared, showing loss of functional connectivity and reduced cognitive readiness. The theta-to-alpha ratio, a useful marker, can climb from a healthy range between 0.5 and 0.8 to an unhealthy range between 1.5 and 2.0 or higher, indicating cognitive decline. Beta activity, which is be between 13 hertz 30 hertz, which reflects active thinking and sensory-motor integration, also tends to decrease in later stages of the disease. The P300 wave, used to measure attention and working memory, is also significantly altered in Alzheimer's. In healthy adults, the P300 latency is typically between 300 milliseconds and 350 milliseconds and amplitude is between 10 microvolts and 20 microvolts. In individuals with Alzheimer's, P300 latency may increase to between 400 milliseconds and 500 milliseconds, and amplitude often decreases to below 8 microvolts, reflecting slower information processing and impaired stimulus recognition. These changes become more pronounced as the disease advances, and may correlate with performance on memory and attention tasks. Brainwave sensor 1400 data from the isolation therapy device 50 may include EEG data and may be used to monitor disease progression. For example, over 6-12 months, a patient might show: Alpha power declining from 22 microvolts squared to 15 microvolts squared. Theta power increasing from 28 microvolts squared to 36 microvolts squared. The theta/alpha ratio rising from 1.27 to 2.4. P300 latency slowing from 380 milliseconds to 460 milliseconds, while amplitude drops from 9 microvolts to 5 microvolts.

In a third example, the isolation therapy device 50 may be used to detect, monitor, and/or treat symptoms associated with autism spectrum disorder (ASD). EEG (electroencephalography) offers valuable insight into the neural underpinnings of autism by measuring real-time brainwave activity. Many symptoms of autism spectrum disorder (ASD) are associated with distinctive patterns in theta, alpha, beta, and gamma brainwaves, which can be observed during rest or specific cognitive tasks. In individuals with autism, EEG often shows increased theta activity which is between 4 hertz and 8 hertz in frontal regions, especially during tasks involving attention or sensory input. This elevated theta may be linked to inattention or cognitive slowing. At the same time, alpha waves which is between 8 herts and 13 hertz, typically associated with relaxed wakefulness and sensory regulation, are often reduced or dysregulated, especially in the visual and auditory cortices. This may contribute to sensory hypersensitivityor difficulty filtering stimuli. Frontal alpha asymmetry, a pattern where one side of the brain shows stronger alpha than the other, is also commonly seen in ASD. Greater right-sided alpha (meaning less activation on the right) is often associated with withdrawal or anxiety symptoms, which are common in autism. In social and language tasks, EEG may show reduced mu suppression (a type of alpha rhythm over motor areas), which is thought to reflect impairments in the mirror neuron system—a mechanism involved in understanding others' actions and emotions. Similarly, beta and gamma activity which is between 13 hertz and 100 hertz, which support focused attention and language processing, may be lower or less synchronized in individuals with ASD, contributing to communication and social interaction difficulties. Example Trends in EEG for Autism Frontal theta power may be elevated (>30 microvolts squared). Occipital alpha power may be reduced (<15 microvolts squared). Theta/alpha ratio may be high (>2.0). Mu rhythm suppression may be reduced or absent during observation of movement. P300 ERP may be delayed or lower amplitude during attention or face recognition tasks. Gamma synchrony may be reduced during language or emotional processing tasks These EEG markers may support diagnosis, track symptom progression, and guide interventions like neurofeedback or behavioral therapy tailored to individual brain activity patterns.

1. An isolation therapy device 50 with a three layer construction comprising:
an adjustable soft compression layer 100 where compression is controlled via a ratchet system 107 and knob 106;
an outermost layer 200 formed from stretchable fabric;
an innermost layer 300 formed from padded, washable fabric;
a sound reduction system 400 integrated within the device to reduce auditory stimuli;
a sight reduction system 700 removably attachable to the device and configured to block light;
and a smell system 1000 containing swappable scent inserts

2. The isolation therapy device 50 of claim 1, wherein brainwave sensors 1205 are adhered to the suspension straps 105.

3. The isolation therapy device 50 of claim 2, wherein at least three brainwave sensors 1205 are adhered to the suspension straps 105 such that they are positioned to be located in the O1, O2, and Oz, Electrode Placement System positions and measure alpha waves.

4. The isolation therapy device 50 of claim 3, wherein at least three brainwave sensors 1205 are adhered to the suspension straps 105 such that they are positioned to be located in three separate positions selected from the Fz, F3, F4, Cz, C3, and C4 Electrode Placement System positions and measure theta waves.

5. The isolation therapy device 50 of claim 4, that measures alpha power, theta power, theta/alpha ratio and P300 latency.

6. The isolation therapy device 50 of claim 5, that is used to detect, monitor and treat symptoms from a concussion.

7. The isolation therapy device 50 of claim 5, that is used to detect, monitor and treat symptoms from cognitive decline.

8. The isolation therapy device 50 of claim 5, that is used to detect, monitor and treat symptoms from anxiety.

9. The isolation therapy device 50 of claim 5, that is used to detect, monitor and treat symptoms from autism spectrum disorder.

10. The isolation therapy device 50 of claim 1, wherein at least two vibration coins 1206 are adhered to the suspension straps 105.

11. The isolation therapy device 50 of claim 10, wherein brainwave sensors 1205 are adhered to the suspension straps 105.

12. The isolation therapy device 50 of claim 11, wherein at least three brainwave sensors 1205 are adhered to the suspension straps 105 such that they are positioned to be located in the O1, O2, and Oz, Electrode Placement System positions and measure alpha waves.

13. The isolation therapy device 50 of claim 12, wherein at least three brainwave sensors 1205 are adhered to the suspension straps 105 such that they are positioned to be located in three separate positions selected from the Fz, F3, F4, Cz, C3, and C4 Electrode Placement System positions and measure theta waves.

14. The isolation therapy device 50 of claim 13, that measures alpha power, theta power, theta/alpha ratio and P300 latency.

15. The isolation therapy device 50 of claim 14, that is used to detect, monitor and treat symptoms from a concussion.

16. The isolation therapy device 50 of claim 15, that is used to detect, monitor and treat symptoms from cognitive decline.

17. The isolation therapy device 50 of claim 16, that is used to detect, monitor and treat symptoms from anxiety.

18. The isolation therapy device 50 of claim 17, that is used to detect, monitor and treat symptoms from autism.

19. The isolation therapy device 50 of claim 1, wherein innermost layer 300 is removable and can be connected to the soft compression layer 100.

20. The isolation therapy device 50 of claim 18, wherein innermost layer 300 is removable and has integrated gel-like pads that can be cooled in the refrigerator and/or freezer, as well as, heated in the microwave.
